(19) United States
(12) Patent Application Publication
(10) Pub. No.: US 2014/0066373 A1
(43) Pub. Date: Mar. 6, 2014

(54) TREATING DIABETES WITH OXYTOCIN OR OXYTOCIN ANALOGS

(71) Applicant: Dongsheng Cai, Larchmont, NY (US)

(72) Inventor: Dongsheng Cai, Larchmont, NY (US)

(73) Assignee: Albert Einstein College of Medicine of Yeshiva University, Bronx, NY (US)

(21) Appl. No.: 13/975,753

(22) Filed: Aug. 26, 2013

Related U.S. Application Data
(60) Provisional application No. 61/694,487, filed on Aug. 29, 2012.

(51) Int. Cl. A61K 38/08

(52) U.S. Cl. CPC A61K 38/08
USPC 514/7.3; 514/6.9

(57) ABSTRACT

Methods are provided of treating or preventing diabetes in a subject comprising administering to the subject an amount of oxytocin or an oxytocin analog. Methods are also provided of increasing insulin secretion in a subject comprising administering to the subject an amount of oxytocin or an oxytocin analog.

### CROSS-REFERENCE TO RELATED APPLICATIONS

This application claims benefit of U.S. Provisional Application No. 61/694,487, filed Aug. 29, 2012, the contents of which are hereby incorporated by reference.

### STATEMENT OF GOVERNMENT SUPPORT

This invention was made with government support under grant numbers R01 DK078750 and RO1 AG031774, awarded by the National Institutes of Health. The government has certain rights in the invention.

### BACKGROUND OF THE INVENTION

Throughout this application various publications are referred to in parentheses by number. The disclosures of these publications, and all patents and patent application publications and books referred to herein, are hereby incorporated by reference in their entirety into the subject application to more fully describe the art to which the subject invention pertains.

Developing peptide therapeutics for diabetes is a relatively new concept, largely prompted by the discovery of the anti-hyperglycemic action of glucagon-like peptide-1 (GLP-1), an endogenous gut hormone secreted by intestinal L cells upon detection of intestinal nutrients. Different from conventional anti-diabetic medications which control hyperglycemia by reducing hepatic glucose output, increasing peripheral glucose uptake, or forcefully opening pancreatic β cell membrane channels to trigger insulin release, GLP-1 agonists control hyperglycemia by stimulating insulin secretion and suppressing glucagon secretion in a glucose-dependent manner. In addition, the anti-obesity effect of GLP-1 additionally contributes to general improvement of obesity-related diabetes (7).

Subsequently, dipeptidyl peptidase-4 (DPP-4) inhibitors which prolong endogenous GLP-1 effects have been developed as a type of peptidyl anti-diabetic medication (2). Recently, bariatric surgery has emerged as a surgical treatment option for diabetes due to its endocrine modifying effects, including the promotion of GLP-1 secretion together with other modulations such as a decrease in ghrelin secretion and an increase in peptide YY secretion both of which reduce appetite and thus obesity (32). Notably, the gastric bypass type bariatric procedures can immediately (as soon as 15 days post-surgery) lower blood sugar levels of type 2 diabetes subjects before they start to lose weight (23; 33), and one mechanism underlying this rapid anti-hyperglycemic action has been related to intestinal nutrient sensing-triggered central adjustment of glucose production (6). Overall, GLP-1, DPP-4 inhibitors or bariatric surgery all point to the promising new direction of endocrine peptide related anti-diabetic therapeutics which independently or complementarily relies on obesity control. These recent advances call for explorations into new types of anti-diabetic peptide medicines which could have potentials of overcoming both insulin secretion defect and insulin resistance to comprehensively control diabetes (16).

The current invention identifies a novel treatment for diabetes based on oxytocin.

### SUMMARY OF THE INVENTION

A method is provided for treating or preventing diabetes in a subject comprising administering to the subject an amount of oxytocin or of an oxytocin analog effective to treat or prevent diabetes. Preventing diabetes, as used herein, is attenuating one or more symptoms of diabetes or parameters by which diabetes is assessed.

Also provided is a method for increasing insulin secretion in a subject comprising administering to the subject an amount of oxytocin or of an oxytocin analog effective to increase pancreatic insulin secretion. Increasing insulin secretion, as used herein, means increasing insulin secretion above the level of insulin secretion in the absence of administration of the oxytocin or of the oxytocin analog.

Also provided is oxytocin or an oxytocin analog for treating or preventing diabetes in a subject.

Also provided is oxytocin or an oxytocin analog for increasing insulin secretion in a subject.

Also provided is a method of improving pancreatic beta cell function in a diabetic subject comprising administering to the subject an amount of oxytocin or of an oxytocin analog effective to improve pancreatic beta cell function.

Also provided is a method of improving pancreatic beta cell function in a pre-diabetic subject having impaired pancreatic beta cell function comprising administering to the subject an amount of oxytocin or of an oxytocin analog effective to improve pancreatic beta cell function.

Additional objects of the invention will be apparent from the description which follows.

### BRIEF DESCRIPTION OF THE DRAWINGS

FIG. 1A-1D. Effects of oxytocin treatment on glucose metabolic profile of prediabetic mice. A-D: C57BL/6 mice were maintained on HFD feeding for 2 months from weaning to develop prediabetic condition, and then received acute intra-third ventricle administration of oxytocin or vehicle. The effects of drug treatment on glucose metabolism and energy balance were evaluated by glucose tolerance test (GTT) (A and B), fasting blood insulin levels (C) and body weight (D). A-D: *P<0.05; n=5-8 (A, B and D) and n=4-5 (C) mice per treatment group. Error bars represent mean±SE. OXT: oxytocin; AUC: area under curve.

FIGS. 2A-2D. Effects of [Ser4, Ile8]-oxytocin (SEQ ID NO:2) treatment on glucose metabolic profile of prediabetic mice. A-D: C57BL/6 mice were maintained on HFD feeding for 2 months from weaning to develop prediabetic condition, and then received acute intra-third ventricle administration of [Ser4, Ile8]-oxytocin or vehicle. The effects of drug treatment on glucose metabolism and energy balance were evaluated by glucose tolerance test (GTT) (A and B), fasting blood insulin levels (C) and body weight (D). A-D: *P<0.05, **P<0.01, ***P<10-3; n=5-8 (A, B and D) and n=4-5 (C) mice per treatment group. Error bars represent mean±SE. [Ser4, Ile8]OxT: [Ser4, Ile8]-oxytocin; AUC: area under curve.

FIG. 3A-3D. Effects of [Asu1,6]-oxytocin (SEQ ID NO:3) treatment on glucose metabolic profile of prediabetic mice. A-D: C57BL/6 mice were maintained on HFD feeding for 2 months to develop prediabetic condition, and then received acute intra-third ventricle administration of [Asu1,6]-oxytocin or vehicle. The effects of drug treatment on glucose metabolism and energy balance were evaluated by glucose tolerance test (GTT) (A and B), fasting blood insulin levels (C) and body weight (D). A-D: *P<0.05; n=5-8 (A, B and D) and n=4-5 (C) mice per treatment group. Error bars represent mean±SE. [Asu1,6]OXT: [Asu1,6]-oxytocin; AUC: area under curve.

FIG. 4A-4E. Effects of oxytocin treatment on glucose metabolic profile of Streptozotocin (STZ)-induced diabetic mice. A-E: C57BL/6 mice under normal chow feeding received 7-day intra-third ventricle administration of oxytocin or vehicle, and were subjected to STZ induction of diabetes on the final 4 days of oxytocin treatment. The effects of oxytocin pretreatment on glucose metabolism and energy balance were evaluated by glucose tolerance test (GTT) (A and B), fasting blood insulin levels (C), first-phase and second-phase glucose-stimulated insulin release (GSIS) (D) and body weight (E). A-E: *P

<0.05, **P<0.01; n=4-6 mice per treatment group. Error bars represent mean±SE. OXT: oxytocin; AUC: area under curve.

FIG. 5A-5E. Effects of [Ser4, Ile8]-oxytocin treatment on glucose metabolic profile of STZ-induced diabetic mice. A-E: C57BL/6 mice under normal chow feeding received 7-day intra-third ventricle administration of [Ser4, Ile8]-oxytocin or vehicle, and were subjected to STZ induction of diabetes during the final 4 days of [Ser4, Ile8]-oxytocin treatment. The effects of [Ser4, Ile8]-oxytocin pretreatment on glucose metabolism and energy balance were evaluated by glucose tolerance test (GTT) (A and B), fasting blood insulin levels (C), first-phase and second-phase glucose-stimulated insulin release (GSIS)

(D) and body weight (E). A-E: *P<0.05, **P<0.01; n=4-6 mice per treatment group. Error bars represent mean±SE. [Ser4, Ile8]OxT: [Ser4, Ile8]-oxytocin; AUC: area under curve.

FIG. 6A-6E. Effects of [Asu1,6]-oxytocin treatment on glucose metabolic profile of STZ-induced diabetic mice. A-E: C57BL/6 mice under normal chow feeding received acute 7-day intra-third ventricle administration of [Asu1,6]-oxytocin or vehicle, and were subjected to STZ induction of diabetes during the final 4 days of [Asu1,6]-oxytocin treatment. The effects of [Asu1,6]-oxytocin pretreatment on glucose metabolism and energy balance were evaluated by glucose tolerance test (GTT) (A and B), fasting blood insulin levels (C), first-phase and second-phase glucose-stimulated insulin release (GSIS) (D) and body weight (E). A-E: *P<0.05; n=4-6 mice per treatment group; control group and STZ-induction group were shared with parallel experiments in FIG. 5. Error bars represent mean±SE. [Asu1,6]OXT: [Asu1,6]-oxytocin; AUC: area under curve.

### DETAILED DESCRIPTION OF THE INVENTION

A method is provided for treating or preventing diabetes in a subject comprising administering to the subject an amount of oxytocin or of an oxytocin analog effective to treat or prevent diabetes. Preventing diabetes, as used herein, is attenuating one or more symptoms of diabetes or parameters by which diabetes is assessed.

Also provided is a method for increasing insulin secretion in a subject comprising administering to the subject an amount of oxytocin or of an oxytocin analog effective to increase pancreatic insulin secretion. Increasing insulin secretion, as used herein, means increasing insulin secretion above the level of insulin secretion in the absence of administration of the oxytocin or of the oxytocin analog.

In an embodiment of the methods, the oxytocin or oxytocin analog is administered so as to permit the oxytocin or oxytocin analog to enter the central nervous system of the subject

In an embodiment of the methods, the amount of oxytocin or of the oxytocin analog is effective to reduce insulin-resistance.

In an embodiment of the methods, the amount of oxytocin or of the oxytocin analog is effective to reduce glucose-intolerance.

In an embodiment of the method for treating diabetes, the diabetes is type-1 diabetes. In an embodiment of the method for increasing insulin secretion, the subject has diabetes and the diabetes is type-1 diabetes. In an embodiment of the method for treating diabetes, the diabetes is type-2 diabetes. In an embodiment of the method for increasing insulin secretion, the subject has diabetes and the diabetes is type-2 diabetes. In an embodiment of the method for treating diabetes, the diabetes is associated with insulin resistance and/or an insulin secretion defect. In an embodiment of the method for increasing insulin secretion, the subject has diabetes and the diabetes is associated with insulin resistance and/or an insulin secretion defect.

In an embodiment of the methods, the oxytocin analog is administered. In an embodiment of the methods, the analog is [Ser4,Ile8]-oxytocin (isotocin) or [Asu1,6]-oxytocin (deamino-dicarba-oxytocin).

In an embodiment of the methods, the oxytocin is administered.

In an embodiment of the methods, the subject is not obese. In an embodiment of the methods, the subject is obese. In an embodiment of the methods, the subject is not overweight. In an embodiment of the methods, the subject is overweight.

In an embodiment of the methods, the oxytocin or oxytocin analog is administered directly into the central nervous system of the subject. In an embodiment of the methods, the oxytocin or oxytocin analog is administered intrathecally. In an embodiment of the methods, the oxytocin or oxytocin analog is administered directly into the central nervous system of the subject from an oxytocin-releasing implant in the subject. In an embodiment of the methods, the implant is in the central nervous system of the subject. In an embodiment of the methods, the oxytocin or oxytocin analog is administered intranasally. In an embodiment of the methods, the oxytocin or oxytocin analog is administered peripherally to the subject.

In an embodiment of the methods, the administered oxytocin or oxytocin analog stimulates endogenous production and/or release of native oxytocin in the subject. In an embodiment of the methods, the administered oxytocin or oxytocin analog stimulates endogenous production and/or release of native oxytocin in the central nervous system of the subject.

In an embodiment of the methods, the oxytocin analog is a peptide analog.

Also provided is oxytocin or an oxytocin analog for treating or preventing diabetes in a subject.

Also provided is oxytocin or an oxytocin analog for increasing insulin secretion in a subject.

In an embodiment, the oxytocin or oxytocin analog is formulated so as to be suitable for administration so as to permit the oxytocin or oxytocin analog to enter the central nervous system of the subject. In an embodiment, the oxytocin or of the oxytocin analog is an amount effective to reduce insulin-resistance. In an embodiment, the oxytocin or of the oxytocin analog is an amount effective to reduce glucose-intolerance.

In an embodiment for treating diabetes, the diabetes is type-1 diabetes. In an embodiment for increasing insulin secretion, the subject has diabetes and the diabetes is type-1 diabetes. In an embodiment, the subject has type-1 diabestes and is non-obese. In an embodiment, the subject has type-1 diabestes and is not overweight. In an embodiment for treating diabetes, the diabetes is type-2 diabetes. In an embodiment for increasing insulin secretion, the subject has diabetes and the diabetes is type-2 diabetes. In an embodiment for treating diabetes, the diabetes is associated with insulin resistance and/or an insulin secretion defect. In an embodiment for increasing insulin secretion, the subject has diabetes and the diabetes is associated with insulin resistance and/or an insulin secretion defect.

In an embodiment, the oxytocin analog is to be administered. In an embodiment, the analog is to be administered. In an embodiment, the analog is [Ser4,Ile8]-oxytocin (isotocin) or [Asu1,6]-oxytocin (deamino-dicarba-oxytocin). In an embodiment, the analog is any one of the oxytocin analogs listed herein. In an embodiment, the analog is a peptide analog In an embodiment, the oxytocin is to be administered.

In an embodiment, the subject is not obese. In an embodiment, the subject is obese. In an embodiment, the subject is not overweight. In an embodiment, the subject is overweight.

In an embodiment, the oxytocin or oxytocin analog is formulated for administration directly into the central nervous system of the subject. In an embodiment, the oxytocin or oxytocin analog is formulated for administration intrathecally. In an embodiment, the oxytocin or oxytocin analog is formulated for administration directly into the central nervous system of the subject. In an embodiment, the oxytocin or oxytocin analog is formulated for administration from an oxytocin-releasing implant in the subject. In an embodiment, the implant is in the central nervous system of the subject. In an embodiment, the oxytocin or oxytocin analog is formulated for administration intranasally. In an embodiment, the oxytocin or oxytocin analog is formulated for administration peripherally to the subject.

In an embodiment, the administered oxytocin or oxytocin analog stimulates endogenous production and/or release of native oxytocin in the subject. In an embodiment, the administered oxytocin or oxytocin analog stimulates endogenous production and/or release of native oxytocin in the central nervous system of the subject.

In an embodiment, the oxytocin analog is a peptide analog.

As used herein, obesity is characterized by the subject having a body mass index of 30.0 or greater (and thus includes the states of significant obesity, morbid obesity, super obesity, and super morbid obesity). In regard to gender, women with over 30% body fat are considered obese, and men with over 25% body fat are considered obese. In an embodiment of the methods of the invention the subject is not obese. As used herein, an overweight subject is defined as a subject having body mass index of from 25.0 to 29.9. In an embodiment of the methods of the invention the subject is not overweight. In an embodiment, a subject is considered overweight when the subject has a body mass index (BMI) of 25-29.9. In an embodiment, a subject is considered obese when the subject has a BMI of 30 or greater. In an embodiment, a subject is considered non-obese when they have a BMI of less than 30. In a further embodiment, a subject is considered non-obese and not overweight when they have a BMI of less than 25.

Any acceptable route of administration of the active compounds described herein can be used. Non-limiting examples include oral, lingual, sublingual, buccal, parenteral, intravenous, intrarterial, intraperitoneal, intrabuccal, intrathecal, intracerebroventricular, nasal administration or administration via implant within the subject can be effected without undue experimentation by means well known in the art.

Pharmaceutical formulations comprising the active compounds described herein (i.e. oxytocin or an oxytocin analog) can be employed in the methods. Pharmaceutical formulations are readily produced, and can comprise, for example, an inert diluent, an edible carrier. The compositions may be suitably, enclosed. for example in gelatin capsules or compressed into tablets. For the purpose of oral therapeutic administration, the pharmaceutical compositions of the present invention may be incorporated with excipients. Tablets, pills, capsules, troches and the like may also contain binders, recipients, disintegrating agent, lubricants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and nontoxic in the amounts used.

For nasal administration, including for administration via the olfactory epithelia in an embodiment of the present invention, the active compound (i.e. oxytocin or an oxytocin analog) or a composition comprising such is administered to the mucous membranes of the nasal passage or nasal cavity of the patient. Pharmaceutical compositions for nasal administration include compositions prepared by well-known methods to be administered, for example, as a nasal spray, nasal drop, suspension, gel, ointment, cream or powder. Administration of the active compound or a composition comprising such may also take place using a nasal tampon or nasal sponge.

For administration parenterally, or peripherally, such as, for example, by intravenous, intramuscular, intrathecal or subcutaneous injection, administration can be accomplished by incorporating the active compound (i.e. oxytocin or an oxytocin analog) or a composition comprising such of the present invention into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as for example, benzyl alcohol or methyl parabens, antioxidants such as for example, ascorbic acid or sodium bisulfate and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials

The active compound or a composition comprising such as employed in the present invention can be administered via an implant, such as a surgically implanted polymer, which polymer is impregnated with the active compound or a composition comprising such. In an embodiment, such a polymer is implanted in the central nervous system of the subject. In an embodiment, such a polymer is implanted in the subject but outside the central nervous system of the subject. Administration of the active compounds described herein can also be via a surgical implant of a drug-releasing polymer into the central nervous system of the subject. For examples of such see Asthagiri et al., Clinical Neurosurgery 55:27-34, (2008), hereby incorporated by reference.

Administration of the active compounds described herein, for example in the methods described herein, can also be effected by injection or cannulation into the central nervous system of the subject. Administration of the active compounds described herein, for example in the methods described herein, can also be achieved via nasal delivery, including through the olfactory epithelium.

In an embodiment, the oxytocin is mammalian. In an embodiment the oxytocin is recombinant oxytocin having the amino acid sequence of human oxytocin. In an embodiment the oxytocin has the amino acid sequence CYIQNCPLG-NH₂ (SEQ ID NO:1), with, in an embodiment, the two cysteines are linked by a disulfide bridge. In an embodiment the oxycotin is an octapeptide oxytocin disulfide.

In an embodiment, the oxytocin, or an analog thereof, is administered to the subject a plurality of times over a predetermined time period. In an embodiment the oxytocin, or an analog thereof, is administered to the subject daily, weekly, bi-weekly, monthly or bi-monthly. In an embodiment, the oxytocin, or an analog thereof, is administered to the subject continuously (for example, via in implant) over a predetermined time period.

In an embodiment, the oxytocin, or analog thereof, is administered directly to the central nervous system of the subject. In an embodiment, the oxytocin, or analog thereof, is administered directly into a paraventricular nucleus of the subject or a third ventricle of the subject. In an embodiment, the oxytocin, or analog thereof, is administered intracerebroventricularly, or intrathecally, or is administered via a polymer implant impregnated with the oxytocin or the analog thereof.

In an embodiment, the oxytocin analog is a peptide analog. As used herein, a peptide analog is a peptide, preferably a nonapeptide, which may comprise modifications of the consitutent amino acid residues thereof, and possess one or more oxytocin activities, but not necessarily at the same quantitiative extent as that of oxytocin. In an embodiment, the oxytocin analog has one or more of the following activities: a) reduces or reverses insulin resistance associated with dietary obesity in a subject, b) reduces or reverses glucose intolerance associated with dietary obesity in a subject, or c) improves beta cell function in a STZ-induced diabetic mouse.

In an embodiment, the oxytocin analog is 1-(3-mercaptopropanoic acid)-2-(O-ethyl-D-tyrosine)-4-L-threonine-8-L-ornithine-oxytocin or is 1-[4-[(1-Acetyl-4-piperidinyl)oxy]-2-methoxybenzoyl]-4-(2-oxo-2H-3,1-benzoxazin-1(4H)-yl)piperidine. In an embodiment, the oxytocin analog is administered directly to the central nervous system of the subject.

Examples of other oxytocin analogs (and their respective CAS registry nos.) which, in an embodiment, can be employed in the methods described herein are set forth below in the format of oxytocin analog; CAS Registry No.
(1-(2-Hydroxy-3-mercaptopropionic acid))-4-thr-7-gly-oxytocin; 60786-60-9
(8alpha-Hydroxyisocaproic acid)oxytocin; 75607-76-0
1-(1-Mercaptocyclohexaneacetic acid)-2-D-tryptophan-4-L-isoleucine-8-L-argininevasopressin; 130155-44-1
1-(1-Mercapto-cyclohexaneacetic acid)-2-(oet-tyr)-8-orn-oxytocin; 77327-46-9
1-(1-Mercaptocyclohexaneacetic acid)-4-L-threonine-oxytocin; 70056-23-4
1′-(1′-Methyl-4′-thiopiperidine)acetic acid oxytocin; 113789-44-9
1′-(1′-Thio-4′-methylcyclohexane)acetic acid oxytocin; 113789-43-8
1-(2-Hydroxy-3-mercaptopropionic acid) oxytocin; 35924-96-0
1-(3-Mercaptopropanoic acid)-8-arg-vasotocin; 38679-66-2
1-(beta-Mercapto(beta,beta-cyclopentamethylene)propionic acid)-2-phe(Me)-4-thr-8-orn-oxytocin; 110220-69-4
1-(beta-Mercapto-beta,beta-cyclopentamethylenepropionic acid)-2-tyr(ome)-8-orn-oxytocin; 77327-45-8
1-(beta-Mercapto-beta,beta-cyclopentamethylenepropionic acid)-8-orn-oxytocin; 77327-42-5
1-(beta-Mercapto-beta,beta-diethylpropionic acid)-2-(oet-tyr)-8-orn-vasotocin; 77327-44-7
1-(N-Maleoyl-11-aminoundecanoyl)cys-oxytocin; 57078-97-4
1-(N-Maleoyl-gly)cys-oxytocin; 57078-96-3
1,6-alpha-Asu-oxytocin; 14317-68-1
1,6-Di-ser-oxytocin; 28278-63-9
10-Glynh2-oxytocin; 21687-84-3
1-alpha-Mercaptoacetic acid-5-iso-asn-oxytocin; 74221-74-2
1-beta-Mercapto-beta, beta-cyclopentamethylenepropionic acid-oxytocin; 55154-85-3
1-beta-Mercapto-beta, beta-diethylpropionic acid-4-leu-oxytocin; 53607-19-5
1-beta-Mercapto-beta,beta-diethylpropionic acid-2-(3,5-dibromo-tyr)-oxytocin; 57292-38-3
1-Butyric-4-L-glutamic-1-carbaoxytocin methyl ester; 72289-65-7
1-Butyric-4-L-glutamic-1-carbaoxytocine; 66714-24-7
1-Deamino-2-trp-4-val-8-orn-oxytocin; 92407-79-9
1-Deamino-2-tyr(ethyl)oxytocin; 77648-79-4
1-Deamino-4-lys(azidobenzoyl)-8-arg-vasotocin; 109798-23-4
1-Deaminopenicillamine-2-phe-4-thr-oxytocin; 72930-66-6
1-Deaminopenicillamine-oxytocin; 6663-74-7
1-Desaminopenicillamyl-2-leu-oxytocin; 92444-08-1
1-Desaminopenicillamyl-2-meo-tyr-4-thr-oxytocin; 70056-25-6
1-Desaminopenicillamyl-2-phe-oxytocin; 72915-15-2
1-Desaminopenicillamyl-4-thr-oxytocin; 60769-46-2
1-Desaminopenicillamyl-8-orn-oxytocin; 72915-15-2
1-Desaminopenicillamyl-meo-2-tyr-oxytocin; 70056-24-5
1-Desamino-thio-9-gly-oxytocin; 43157-27-3
1-Mpa-cyclo(4-glu-8-lys)-oxytocin; 125666-62-8
1-Pen-2-(4-mephe)-4-thr)₈-orn-oxytocin; 106128-84-1
1-Pen-2-phe-4-thr-8-orn-oxytocin; 136429-81-7
1-Penicillamine-oxytocin; 6592-93-4
1-Penicillamyl-2-leu-oxytocin; 68974-28-7
1-Penicillamyl-2-phe-4-thr-oxytocin; 78578-27-5
1-Penicillamyl-4-leu-oxytocin; 57203-07-3
1-Penicillamyl-4-thr-oxytocin; 78578-24-2
1-Penicillamyl-O-2-metyr-oxytocin; 89070-65-5
2-(4-Ethyl-phe)-oxytocin; 24870-58-4
2-(O-Methyl-1-tyrosine)-oxytocin; 2706-70-9
2-Bromoacetylamino-phe-deamino-oxytocin; 67651-40-5
2-L-Dopa-oxytocin; 59845-47-5
2-Mephe-oxytocin; 3714-57-6
2-Nitro-5-azidobenzoyl-gly-oxytocin; 66735-75-9
2-Phe-8-orn-oxytocin; 2480-41-3
2-Trp-oxytocin; 37883-08-2
4-Fluoro-2-phe-oxytocin; 13018-67-2
4-Glu(nhnh2)-oxytocin; 127716-65-8
4-Glu-oxytocin; 4314-67-4
4-Gly-oxytocin; 1976-82-5
4-His-oxytocin; 57735-87-2
4-Homo-ser-oxytocin; 63529-98-6
4-Leu-vasotocin; 39729-33-4
4-L-Threonine-oxytocin; 26995-91-5
4-Ser-tocinoic acid; 53032-97-6
4-Thr-7-gly-oxytocin; 60786-59-6
4-Thr-N-7-meala-oxytocin; 86969-97-3
5-(N(4),N(4)-Dimethyl-asn)-oxytocin; 70232-18-7
5-Asp-oxytocin; 65907-78-0
5-beta-Cyano-ala-oxytocin; 87590-89-4
5-beta-Malamidic acid-oxytocin; 83281-46-3
7-(Azetidine-2-carboxylic acid)oxytocin; 72302-74-0
7-(Thiazolidine-4-carboxylic acid)oxytocin; 59095-56-6
7-Gly-oxytocin; 19748-53-9
7-Sar-oxytocin; 77225-24-2
8-alpha-Hydroxyisocaproic acid)oxytocin-1-desaminopenicillamine; 88508-87-6
8-L-Valine-oxytocin; 3275-87-4
8-Lys-oxytocin; 4273-93-2
8-Trp-oxytocin; 75511-62-5
9 alpha-Aminoacetonitrile-oxytocin; 82031-30-9
9-De-gly-NH2-oxytocin; 4294-07-9
9-Des-gly-2-tyr(oet)-4-thr-8-orn-oxytocin; 151272-15-0
Annetocin; 154445-03-1
Argiprestocin; 113-80-4
Aspartocin; 4117-65-1
Asvatocin; 144334-52-1
Atosiban; 90779-69-4
beta-Mercapto-beta,beta-cyclopentamethylenepropionic acid-2-trp-8-arg-oxytocin; 133851-41-9
Carbetocin; 37025-55-1
Cargutocin; 33605-67-3
Conopressin G; 111317-91-0
Dansyl-8-lys-vasotocin; 138915-83-0
Deamino-(8-alpha-hydroxyisocaproic acid)oxytocin; 71375-94-5
Deamino-1-carba-oxytocin; 20576-70-9
Deamino-6-carba-oxytocin; 30927-32-3
Deamino-8-(N-Me-leu)-oxytocin; 71375-93-4
Demoxytocin; 113-78-0
Desamino-2-(p-fluoro-phe)-oxytocin; 52574-21-7
Dicarbaoxytocin; 4294-09-1
Glumitocin; 10052-67-2
Gly-lys-arg-oxytocin; 90685-16-8
Hydrin 1; 122842-47-1
Hydrin 2; 122842-55-1
Hydroxy-4-thr-oxytocin; 58418-35-2
Isotocin; 550-21-0
L-Prolyl-L-leucylglycylamide; 2002-44-0
Mesotocin; 362-39-0
Methyl oxytocin; 9081-32-7
Nacartocin; 77727-10-7
N-Acetyl-2-O-methyl-tyr-oxytocin; 30750-55-1
N-Acetyloxytocin; 10551-48-1
Oxypressin; 642-35-3
Oxytocin acetate; 6233-83-6
Oxytocin dihydrogen citrate; 74499-03-9
Oxytocin; 50-56-6
Oxytocinoic acid dimethylamide; 73631-36-4
Phasvatocin; 144334-53-2
Pressinoic acid; 35748-51-7
Syntometrine; 37209-62-4
Tocinamide; 13018-33-2
Tocinoic acid; 34330-23-9
Tri-gly-oxytocin; 16639-11-5
Vasotocin; 9034-50-8.

Also provided is a method of improving pancreatic beta cell function in a diabetic subject comprising administering to the subject an amount of oxytocin or of an oxytocin analog effective to improve pancreatic beta cell function. In an embodiment, the subject has impaired pancreatic beta cell function. In an embodiment, the oxytocin or oxytocin analog reduces impairment of pancreatic beta cell insulin secretion. In an embodiment, the oxytocin or oxytocin analog improves glucose intolerance in the diabetic subject. In an embodiment, the diabetic subject has type-1 diabetes. In an embodiment, the diabetic subject does not have type-2 diabetes.

Also provided is a method of improving pancreatic beta cell function in a pre-diabetic subject having impaired pancreatic beta cell function comprising administering to the subject an amount of oxytocin or of an oxytocin analog effective to improve pancreatic beta cell function. In an embodiment, the oxytocin or oxytocin analog reduces impairment of pancreatic beta cell insulin secretion. In an embodiment, the oxytocin or oxytocin analog improves glucose intolerance in the diabetic subject.

In an embodiment of the methods described herein, the subject is mammalian. In a preferred embodiment, the subject is a human.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

Oxytocin is a nine-amino acid peptide hormone existing in many species. Oxytocin (Cys-Tyr-Ile-Gln-Asn-Cys-Pro-Leu-Gly-NH₂) (SEQ ID NO:1) is produced by hypothalamic oxytocin neurons in humans, and released via posterior lobe of pituitary into circulation to act on peripheral targets. Oxytocin was originally best known for its female reproductive functions in labor and lactation (34). More recently, oxytocin was found to play a gender-unrelated role in promoting beneficial behaviors such as social recognition, emotional bonding, trust, love and care (3; 4; 8; 11; 15; 17). Most recently, another aspect of oxytocin function was discovered: consistent with mice genetically deficient of oxytocin or oxytocin receptor developing overeating and obesity (1; 18; 35), chronic central or peripheral oxytocin treatment can decrease obesity (25; 37) or metabolic circadian arrhythmicity (38).

Here it is disclosed that that oxytocin and oxytocin analogs (including [Ser4, Ile8]-oxytocin and [Asu1,6]-oxytocin) have acute anti-diabetic effects, in a body weight-independent manner however, in animal disease models, and these effects are mediated by reversal of insulin resistance and improvement of insulin secretion. These results disclose oxytocin and its selective peptide analogs as a new type of anti-diabetic therapeutic for both type 1 and type 2 diabetes.

### Materials and Methods

Animals. C57BL/6 mice were purchased from Jackson Laboratory. All mice were housed in standard conditions. High-fat diet was purchased from Research Diets, Inc. All animal procedures were approved by the Institutional Animal Care and Use Committee. Mice were subjected to body weight measurement on a daily basis surrounding surgical procedures or drug treatments to monitor physical recovery or the effect of drug treatment on body weight.

Third ventricle cannulation and drug treatment. Third ventricle cannulation procedure was as previously described (39). An ultra-precise small animal stereotactic apparatus (Kopf Instruments) was used to perform implantation of a guide cannula into the third ventricle of anesthetized mice, at the coordinates of 1.8 mm posterior to the bregma and 5.0 mm below the skull surface. Mice were allowed 1-2 weeks for postsurgical recovery. For drug treatment. Oxytocin (Bachem Americas, Inc.), [Ser4, Ile8]-oxytocin (Bachem Americas, Inc.), [Asu1,6]-oxytocin (American Peptide Company, Inc.) or control vehicle were injected via pre-implanted guide cannula into the third ventricle of individually housed mice.

Blood tests. For glucose tolerance test (GTT), overnight-fasted mice received one-time intraperitoneal injection of glucose (2 g/kg body weight), and blood glucose levels were measured using OneTouch Ultra2 (LifeScan, Inc.). Fasting blood insulin levels were measured with Ultra Sensitive Mouse Insulin ELISA kits (Crystal Chem). Glucose-stimulated insulin secretion (GSIS) test was performed as previously described (19). Overnight-fasted mice received one-time intraperitoneal injection of glucose (3 g/kg body weight), and blood insulin levels were measured at 3 min. and 20 min. after the injection.

Statistical analysis. Kolmogorov-Smirnov test was used to determine parametric or nonparametric distribution of data sets. Two-tailed Student's t tests were used for two-group comparisons. ANOVA and appropriate post hoc analyses were used for comparisons involving more than two groups. Data were presented as mean±SEM. P<0.05 was considered statistically significant.

### Results

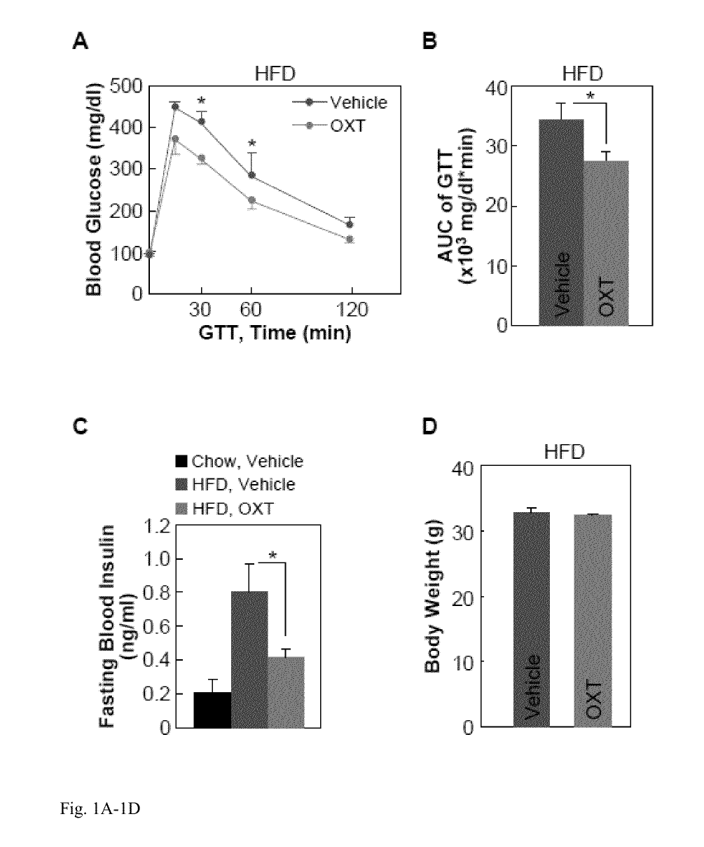

Acute oxytocin therapy reverses insulin resistance in dietary obese mice independently of body weight. A previous study by the present laboratory showed that enhancing in vivo oxytocin function can prevent the development of obesity and other conditions against dietary overnutrition (37). To experimentally address whether oxytocin could have an anti-diabetic action, and independently of its anti-obesity effect, an acute oxytocin therapy paradigm was applied to dietary obese mice to avoid confounding body weight changes, so that the potential anti-diabetes effect of oxytocin could be dissected from its anti-obesity effect. Adult male C57BL/6 mice to 2 months of high-fat diet (HFD) feeding since weaning to develop pre-diabetic glucose intolerance and hyperinsulinemia. Then these mice received third-ventricle implantation of guide cannula followed by postsurgical recovery. Oxytocin was administered via pre-implanted third-ventricle cannula at a dose of 2 μg per injection twice over an overnight fasting period, one at the starting point of fasting and the other at 30 min prior to glucose tolerance test (GTT). Mice of treatment control group received intra-third ventricle vehicle injections. Results showed that mice that received acute oxytocin treatment had significantly improved profiles of glucose intolerance (FIGS. 1, A and B) and fasting blood insulin levels (FIG. 1C). Acute oxytocin treatment did not induce body weight changes in these mice during the overnight period (FIG. 1D). Thus, these results demonstrated that central oxytocin treatment can acutely reverse insulin resistance and glucose intolerance associated with dietary obesity independently of obesity control in mice.

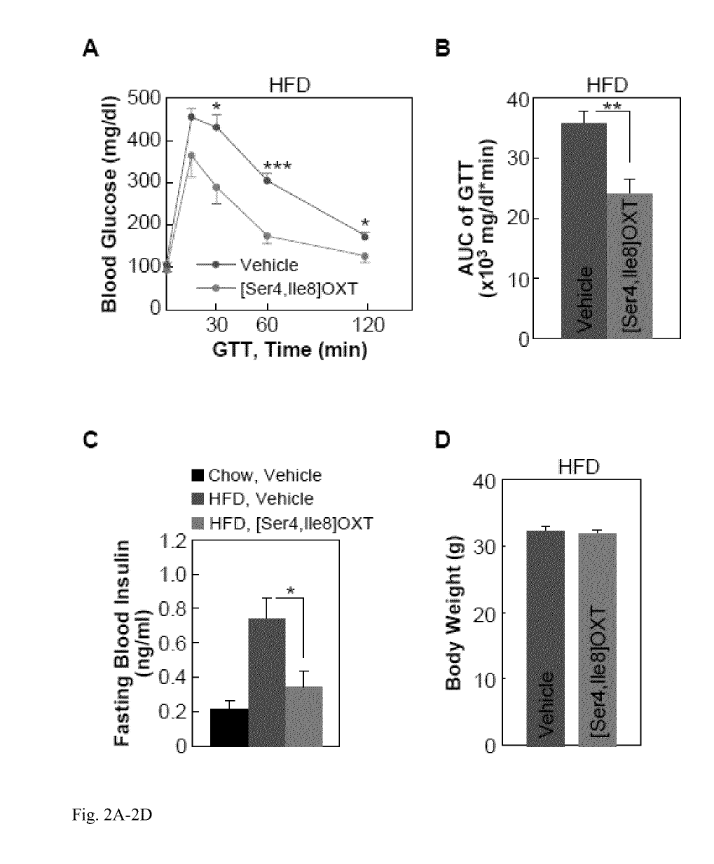

Acute [Ser4, Ile8]-oxytocin therapy improves insulin resistance in dietary obese mice independently of body weight. Having observed the anti-insulin resistance effect of oxytocin, it was next investigated whether oxytocin peptide analogs have similar or stronger treatment effect. The purpose of this experiment was two-fold. First, was to investigate whether the anti-insulin resistance action of oxytocin could be extended to oxytocin analogs. Second, if the first was confirmed, was to investigate more potent oxytocin-like anti-diabetic therapeutics. Existing oxytocin peptide analogs were screened. [Ser4, Ile8]-oxytocin came out of the screen as a good candidate. Using the same 2-month HFD-induced prediabetic mouse model and the same acute therapy paradigm described above, it was found that intra-third ventricle delivery of [Ser4, Ile8]-oxytocin at 2 μg per injection twice over an overnight fasting period not only significantly corrected glucose intolerance (FIGS. 2, A and B) and hyperinsulinemia (FIG. 2C) in mice, but also showed a higher efficacy than oxytocin, since the same dose of [Ser4, Ile8]-oxytocin treatment elicited more pronounced treatment effects than oxytocin. The anti-insulin resistance effect of [Ser4, Ile8]-oxytocin was also independent of body weight, since the acute drug treatment did not incur body weight changes in mice during the overnight period under fasting condition (FIG. 2D). This stronger anti-insulin resistance effect of [Ser4, Ile8]-oxytocin is in line with structure-based theoretical prediction of drug effects. The substitution of glutamine at amino acid position 4 of oxytocin with serine has two direct effects on peptide function—it strengthens the α-helical structure in peptide (13), which leads to an overall structural stabilization, longer drug duration, and better protein binding property with signaling partners; it also increases the hydrophobicity of peptide, which leads to enhanced membrane permeability and improved tissue distribution. The substitution of leucine at amino acid position 8 of oxytocin with isoleucine also increases peptide hydrophobicity and similarly improves drug effect. In summary, [Ser4, Ile8]-oxytocin was identified as another body weight-independent anti-insulin resistance therapeutic agent with stronger effect than oxytocin using the rodent models.

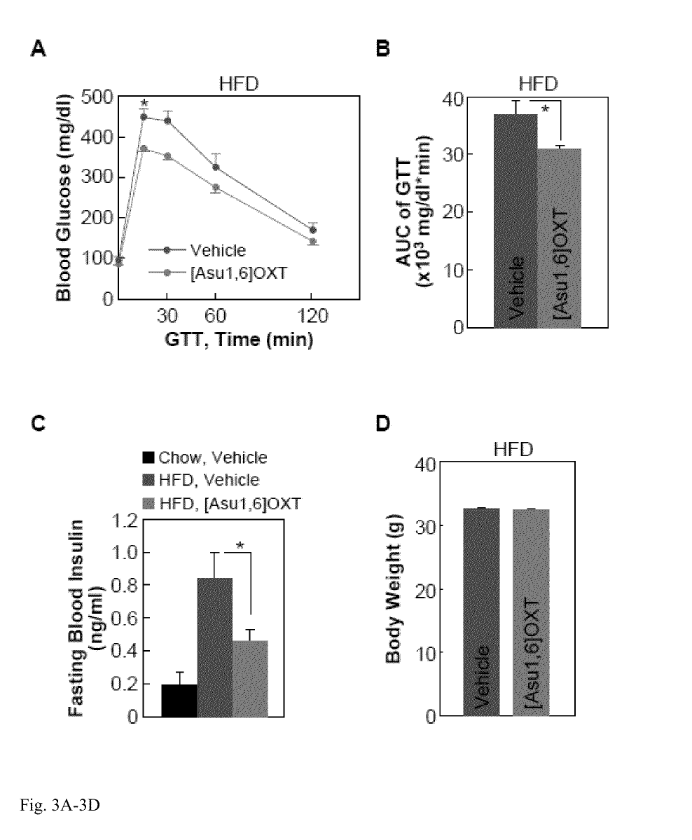

Acute [Asu1,6]-oxytocin therapy improves insulin resistance in dietary obese mice independently of body weight. Another oxytocin analog that was screened with appreciable anti-insulin resistance effect was [Asu1,6]-oxytocin, or called deamino-dicarba-oxytocin, in which both sulfur atoms in the disulfide bridge between Cys1 and Cys6 of oxytocin are replaced by methylene moieties and the terminal amino group is replaced by a hydrogen atom (36). Disulfide bond modification importantly affects plasma stability and metabolic half-life of oxytocin (26), and replacing the terminal amino group by hydrogen by itself increases oxytocin potency in several bioassay systems (12). Using the same dietary obese and prediabetic mouse model as described above, it was found that acute intra-third ventricle administration of [Asu1,6]-oxytocin at 2 μg per injection per day twice over an overnight period improved glucose intolerance (FIGS. 3, A and B) and lowered fasting blood insulin levels (FIG. 3C), and these treatment effects were not accompanied by statistically significant body weight changes in mice (FIG. 3D). However, the treatment effects of [Asu1,6]-oxytocin were slightly weaker than oxytocin (FIG. 1, A-C), which could relate to literature report that the intrinsic activity of [Asu1,6]-oxytocin was only˜70% of natural oxytocin in another context of uterine contractile response (36). Overall, results from [Asu1,6]-oxytocin therapy experiment supported the general paradigm that oxytocin and its selective peptide analogs have anti-insulin resistance actions against overnutrition-induced prediabetic conditions independently of body weight in rodents.

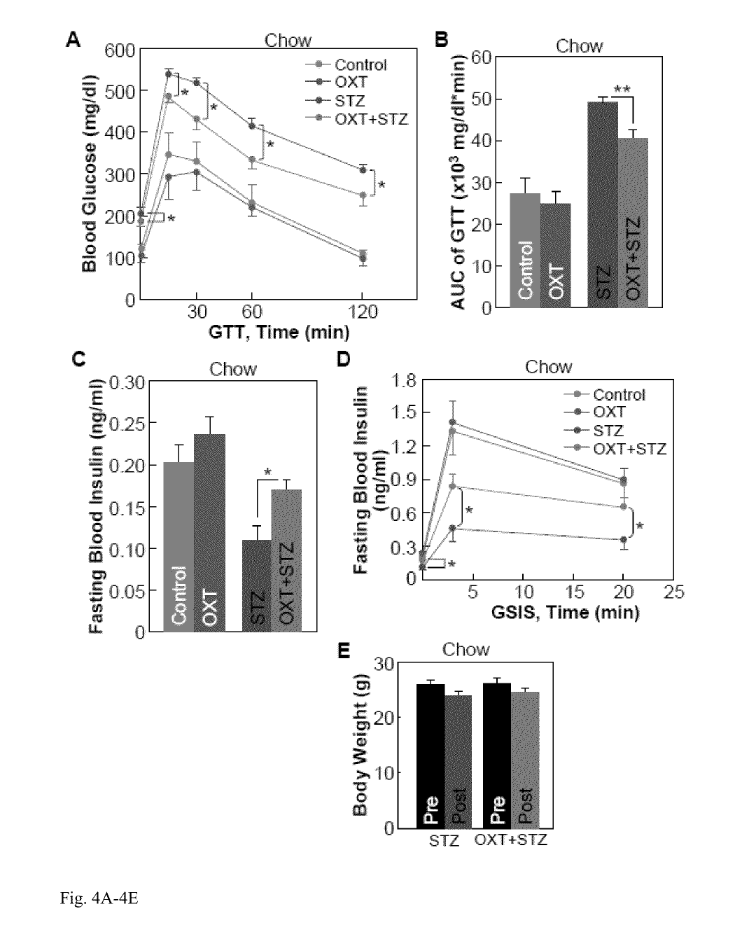

Acute oxytocin therapy significantly improves beta cell function in STZ-induced diabetic mice. Next, it was investigated if oxytocin therapy may treat diabetes by counteracting its main pathological changes of pancreatic beta cell dysfunction and impaired insulin secretion. This hypothesis was suggested by a human study showing that oxytocin gene expression and in vivo function are significantly impaired in type-2 diabetes (14), and by studies showing that central or peripheral administration of oxytocin can stimulate pancreatic insulin secretion in normal rats (5) normal men (29) and type-1 diabetic patients (28). Further suggestive evidence was provided in the recent work showing that in genetic mouse model of enhanced in vivo oxytocin function, pancreatic beta cells were significantly protected from overnutrition-induced diabetic damage (37), though whether the beta cell protection by oxytocin was primary function of this peptide or a secondary effect of obesity control was not differentiated in that study. To assess the potential obesity-unrelated role of oxytocin in protecting beta cell function, a streptozotocin (STZ)-induced diabetes mouse model was employed which recapitulates beta cell damage and insulin secretion impairment in type-1 diabetes, but does not develop energy balance-related disorders such as overeating or obesity. Experimentally, adult C57BL/6 mice on normal chow diet received intra-third ventricle implantation of cannula and postsurgical recovery. Oxytocin pretreatment was given at 1 μg per injection twice per day for 7 consecutive days via pre-implanted cannula, and non-treatment group received vehicle injections. From Day 4 to Day 7 during oxytocin treatment, mice simultaneously received intraperitoneal injections of STZ at 40 mg/kg body weight per day for 4 days to induce diabetes. Blood tests showed that compared to the control STZ-treated mice, oxytocin pretreatment in STZ-treated mice significantly improved glucose intolerance (FIGS. 4, A and B), protected fasting blood insulin levels (FIG. 4C), and potentiated first-phase and second-phase insulin secretions under glucose-stimulated insulin secretion test (FIG. 4D). The anti-diabetic effect of oxytocin pretreatment was not associated with significant changes in body weight (FIG. 4E). Altogether, these results demonstrated that a short-term oxytocin therapy can sufficiently protect pancreatic beta cell function and insulin secretion against STZ-induced diabetic condition in mice.

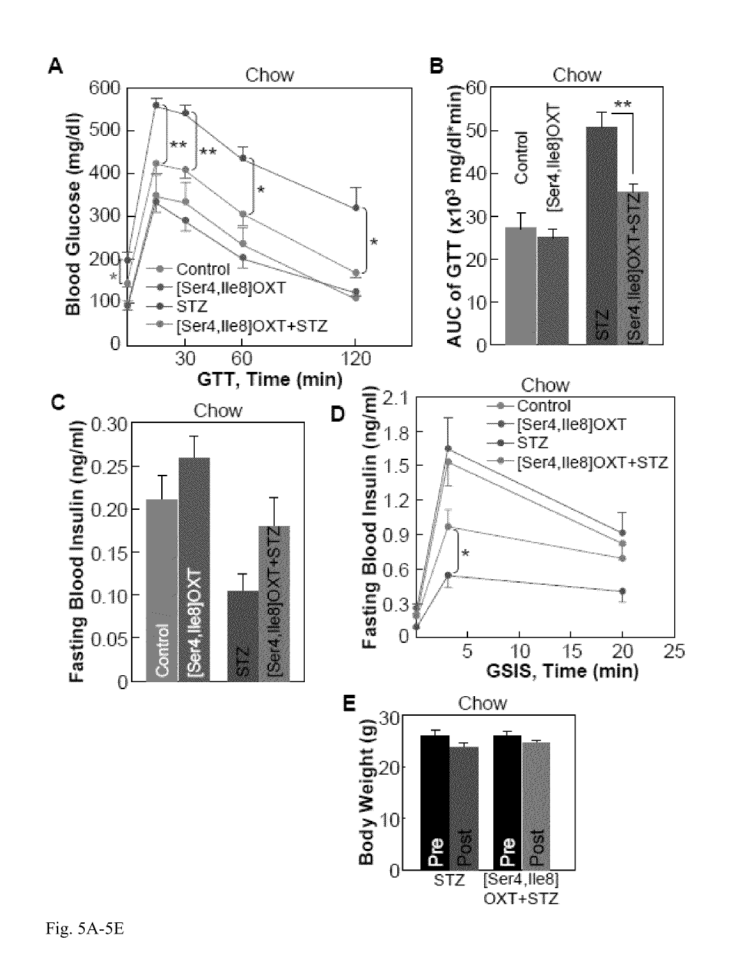

Acute [Ser4, Ile8]-oxytocin therapy significantly improves beta cell function in STZ-induced diabetic mice. Given that [Ser4, Ile8]-oxytocin exhibited anti-insulin resistance action like oxytocin, it was considered if [Ser4, Ile8]-oxytocin may have anti-diabetic and beta cell protective functions similar to oxytocin. The same test strategy of pretreating mice with [Ser4, Ile8]-oxytocin was employed and then evaluating if it could attenuate the extent of STZ-induced beta cell dysfunction and diabetes. Adult chow-fed C57BL/6 mice first received intra-third ventricle injections of [Ser4, Ile8]-oxytocin at 1 mg per injection twice per day for 7 consecutive days. Non-treatment control mice received intra-third ventricle injections of vehicle instead of [Ser4, Ile8]-oxytocin. From Day 4 to Day 7 of [Ser4, Ile8]-oxytocin treatment, both treatment and control groups received intraperitoneal injections of STZ at 40 mg/kg body weight per injection per day for 4 days. The same panel of examinations were performed including glucose tolerance test, fasting blood insulin levels and glucose-stimulated insulin secretion in these mice. [Ser4, Ile8]-oxytocin pretreatment significantly prevented STZ-induced glucose intolerance (FIGS. 5, A and B) and ameliorated circulating insulin insufficiency (FIG. 5C), and the latter was mechanistically attributable to improved beta cell function, since [Ser4, Ile8]-oxytocin treatment group had significantly higher levels of first-phase GSIS than non-treatment group (FIG. 5D). [Ser4, Ile8]-oxytocin treated mice did not involve significant body weight changes in STZ-induced diabetic mice (FIG. 5E). These results demonstrated that acute [Ser4, Ile8]-oxytocin therapy has anti-diabetic actions via protecting pancreatic 13 cell function and improving insulin secretion in mice.

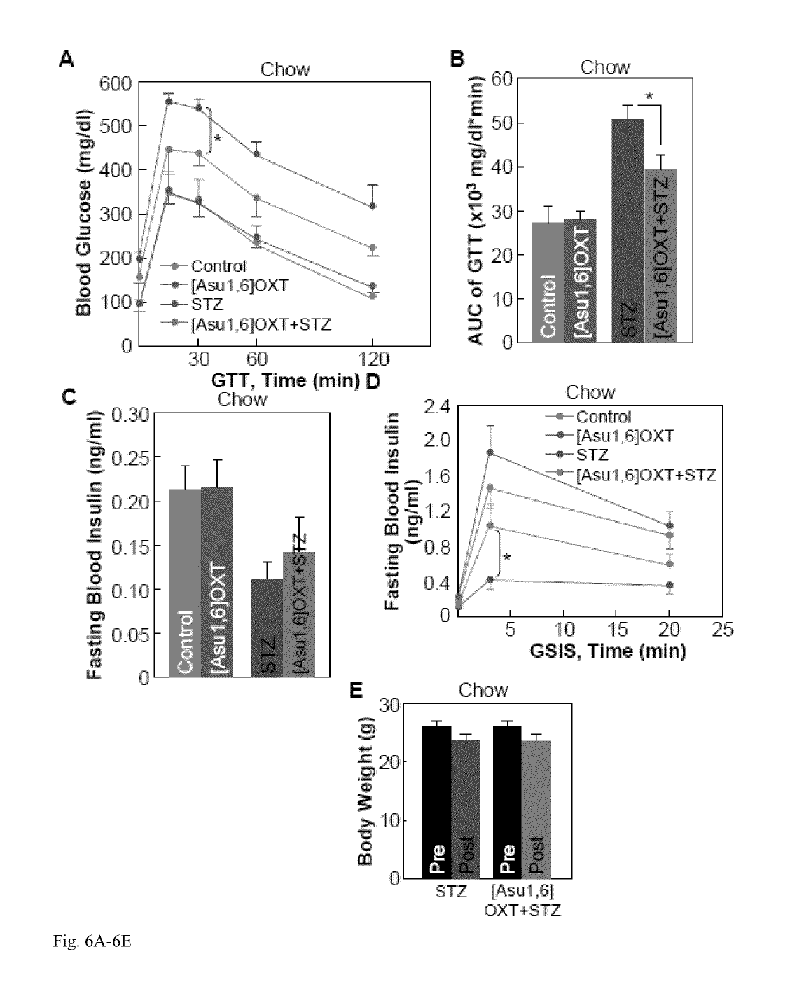

Acute [Asu1,6]-oxytocin therapy significantly improves beta cell function in STZ-induced diabetic mice. Finally [Asu1,6]-oxytocin was tested for potential 13 cell protection and anti-diabetic effects using an STZ-induced diabetic mouse model. [Asu1,6]-oxytocin pretreatment was given to adult chow-fed C57BL/6 mice via intra-third ventricle injections at 1 μg per injection b.i.d. for 7 consecutive days. Vehicle injections were given in parallel to non-treatment control group. From Day 4 to Day 7 of [Asu1,6]-oxytocin treatment, mice were subjected to STZ induction of diabetes at 40 mg/kg body weight per intraperitoneal injection per day for 4 days. Following [Asu1,6]-oxytocin and STZ treatments, mice were evaluated for glucose tolerance test, fasting blood insulin levels, and glucose-stimulated insulin secretion. Results showed that [Asu1,6]-oxytocin pretreatment in STZ-treated mice appreciably improved glucose intolerance (FIGS. 6, A and B), fasting insulin levels (FIG. 6C) and first-phase GSIS (FIG. 6D). The anti-diabetic effects of [Asu1,6]-oxytocin pretreatment was not associated with significant changes in body weight (FIG. 6E). Thus these results showed that acute [Asu1,6]-oxytocin treatment has anti-diabetic effect through protecting and improving 13 cell insulin secretion in mice.

In summary, the results in this study showed that acute pharmacological treatments with peptide compounds oxytocin, [Ser4, Ile8]-oxytocin or [Asu1,6]-oxytocin have anti-diabetic actions. In obesity-induced type-2 prediabetic mice, oxytocin and its analogs sufficiently improve insulin resistance. And in the STZ-induced type-1 diabetic mouse model, which features diabetic beta cell damage and impaired insulin secretion, oxytocin and its analogs can effectively protect beta cells from diabetic impairment of insulin secretion and substantially control glucose intolerance. Notably, both the anti-insulin resistance and anti-diabetic effects of oxytocin, [Ser4, Ile8]-oxytocin and [Asu1,6]-oxytocin occurred without statistically significant body weight changes in the acute therapeutic paradigms, which highlights the anti-diabetic outcome as the primary treatment effects of these peptides, as opposed to secondary effects from obesity control.

### Discussion

Novel anti-insulin resistance therapeutic mechanism of oxytocin and peptide analogs. The anti-insulin resistance action of oxytocin and its analogs discovered by this study elects these peptide compounds as a new type of anti-diabetic medication. Metabolic syndrome-associated type-2 diabetes is known to start from decreased insulin sensitivity in peripheral metabolic tissues, in response to which pancreatic beta cells compensatorily increase insulin production and secretion to maintain a normal blood glycemic profile, until they become functionally and physiologically impaired and uncontrolled hyperglycemia ensues. Thus, early treatment or prevention of insulin resistance is both medically effective and financially economic in combating type-2 diabetes. In this laboratory's earlier work addressing the pathogenic dynamics of metabolic syndrome, it was found that insulin resistance can be induced early on by dietary overnutrition via a neural mechanism in the absence of obesity (31), which argues that there should exist a basis for anti-diabetic medications that target insulin resistance via counteracting its neural induction mechanism. In this study, it was demonstrated that oxytocin and its analogs have anti-insulin resistance effect which is dissociable from body weight control through acute experimental paradigm. This study did not experimentally address the underlying signaling mechanism, but theoretically hypothalamic oxytocin can be released and act locally on the mediobasal hypothalamus—a hypothalamic region that holds neural control over peripheral glucose homeostasis (9; 24), which forms a likely biological basis for oxytocin to participate in neural control of glucose metabolism independently of energy and body weight homeostasis. This anti-insulin resistance action makes oxytocin and its analogs distinct from conventional chemical-based anti-diabetic medicines.

Comprehensive anti-diabetic therapeutic mechanisms of oxytocin and peptide analogs. The comprehensive anti-diabetic mechanism constitutes another forte of oxytocin and its analogs as ideal anti-diabetic therapeutic candidates. In addition to the aforementioned anti-insulin resistance mechanism which primarily targets pre-diabetic stage, earlier work found that enhancing in vivo oxytocin function, either via pharmacological administration of exogenous oxytocin or by genetically removing endogenous oxytocin exocytosis inhibitor, can markedly prevent or reverse the development of dietary obesity and other conditions in mice (37). In addition, the protection of beta cell function against STZ treatment demonstrates the therapeuticuse of oxytocin and its analogs for overt diabetes especially when massive beta cell dysfunction becomes the main pathological culprit. Also, this beta cell protective action against STZ shows oxytocin and its analogs as suitable therapeutic agents for type-1 diabetes, since STZ-induced diabetes is regarded as a type-1 diabetes model.

Beneficial off-target effects of oxytocin and peptide analogs in diabetes treatment. Other biological properties of oxytocin potentially add to the therapeutic advantages of oxytocin and its analogs in treating diabetes. Though initially only recognized for its functions in inducing female uterine contractions and milk let-down, oxytocin is now a well-known hormone for promoting many beneficial social functions such as social recognition, pair bonding, love and care in mammals, and in humans oxytocin can particularly enhance the general mental and emotional well-being (3; 4; 8; 11; 15; 17). In fact, oxytocin has been put into many clinical trials in recent years for conditions such as schizophrenia, autism, anxiety disorder, depression and drug dependence, and the treatment results were positive (10; 22). Meanwhile, epidemiologic studies have found a strikingly high association between diabetes and mental disorders such as schizophrenia, bipolar disorders and severe depression (27). And in one direction, mental dysfunction seems to contribute to diabetes development due to poor lifestyle and nutrition management, usage of appetite-stimulating antipsychotic drugs, and some brain-specific alterations of glucose regulation (20; 21; 30). Hence, it can be inferred that the social-improving functions of oxytocin and its analogs can substantially benefit the diabetic outcome in relevant patient groups via improving lifestyle in general and eating behavior in particular. Also worth mentioning, oxytocin has a major therapeutic advantage over antipsychotic drugs in that oxytocin treatment decreases rather than increases food intake (37).

### REFERENCES

1. Amico J A, Vollmer R R, Cai H M, Miedlar J A and Rinaman L. Enhanced initial and sustained intake of sucrose solution in mice with an oxytocin gene deletion. Am J Physiol Regul Integr Comp Physiol 289: R1798-R1806, 2005.
2. Amori R E, Lau J and Pittas A G. Efficacy and safety of incretin therapy in type 2 diabetes: systematic review and meta-analysis. JAMA 298: 194-206, 2007.
3. Andari E, Duhamel J R, Zalla T, Herbrecht E, Leboyer M and Sirigu A. Promoting social behavior with oxytocin in high-functioning autism spectrum disorders. Proc Natl Acad Sci USA 107: 4389-4394, 2010.
4. Bartz J A, Zaki J, Ochsner K N, Bolger N, Kolevzon A, Ludwig N and Lydon J E. Effects of oxytocin on recollections of maternal care and closeness. Proc Natl Acad Sci USA 107: 21371-21375, 2010.
5. Bjorkstrand E, Eriksson M and Uvnas-Moberg K. Evidence of a peripheral and a central effect of oxytocin on pancreatic hormone release in rats. Neuroendocrinology 63: 377-383, 1996.
6. Breen D M, Rasmussen B A, Kokorovic A, Wang R, Cheung G W and Lam T K. Jejunal nutrient sensing is required for duodenal-jejunal bypass surgery to rapidly lower glucose concentrations in uncontrolled diabetes. Nat Med 2012.
7. Briones M and Bajaj M. Exenatide: a GLP-1 receptor agonist as novel therapy for Type 2 diabetes mellitus. Expert Opin Pharmacother 7: 1055-1064, 2006.
8. Donaldson Z R and Young U. Oxytocin, vasopressin, and the neurogenetics of sociality. Science 322: 900-904, 2008.
9. Elmquist J K, Coppari R, Balthasar N, Ichinose M and Lowell B B. Identifying hypothalamic pathways controlling food intake, body weight, and glucose homeostasis. J Comp Neurol 493: 63-71, 2005.
10. Feifel D. Oxytocin as a potential therapeutic target for schizophrenia and other neuropsychiatric conditions. Neuropsychopharmacology 37: 304-305, 2012.
11. Ferguson J N, Young U, Hearn E F, Matzuk M M, Insel T R and Winslow J T. Social amnesia in mice lacking the oxytocin gene. Nat Genet 25: 284-288, 2000.
12. Ferrier B M, Jarvis D and Du V, V. Deamino-oxytocin. Its isolation by partition chromatography on Sephadex and crystallization from water, and its biological activities. J Biol Chem 240: 4264-4266, 1965.
13. Forood B, Feliciano E J and Nambiar K P. Stabilization of alpha-helical structures in short peptides via end capping. Proc Natl Acad Sci USA 90: 838-842, 1993.
14. Gutkowska J, Broderick T L, Bogdan D, Wang D, Lavoie J M and Jankowski M. Downregulation of oxytocin and natriuretic peptides in diabetes: possible implications in cardiomyopathy. J Physiol 587: 4725-4736, 2009.
15. Ishak W W, Kahloon M and Fakhry H. Oxytocin role in enhancing well-being: a literature review. J Affect Disord 130: 1-9, 2011.
16. Ismail-Beigi F. Clinical practice. Glycemic management of type 2 diabetes mellitus. N Engl J Med 366: 1319-1327, 2012.
17. Kosfeld M, Heinrichs M, Zak P J, Fischbacher U and Fehr E. Oxytocin increases trust in humans. Nature 435: 673-676, 2005.
18. Kublaoui B M, Gemelli T, Tolson K P, Wang Y and Zinn A R. Oxytocin deficiency mediates hyperphagic obesity of Siml haploinsufficient mice. Mol Endocrinol 22: 1723-1734, 2008.
19. Kubosaki A, Nakamura S and Notkins A L. Dense core vesicle proteins IA-2 and IA-2beta: metabolic alterations in double knockout mice. Diabetes 54 Suppl 2: S46-S51, 2005.
20. Leonard B E, Schwarz M and Myint A M. The metabolic syndrome in schizophrenia: is inflammation a contributing cause? J Psychopharmacol 26: 33-41, 2012.
21. Mai Q, Holman C D, Sanfilippo F M, Emery J D and Preen D B. Mental illness related disparities in diabetes prevalence, quality of care and outcomes: a population-based longitudinal study. BMC Med 9: 118, 2011.
22. Meyer-Lindenberg A, Domes G, Kirsch P and Heinrichs M. Oxytocin and vasopressin in the human brain: social neuropeptides for translational medicine. Nat Rev Neurosci 12: 524-538, 2011.
23. Mingrone G, Panunzi S, De G A, Guidone C, Iaconelli A, Leccesi L, Nanni G, Pomp A, Castagneto M, Ghirlanda G and Rubino F. Bariatric surgery versus conventional medical therapy for type 2 diabetes. N Engl J Med 366: 1577-1585, 2012.
24. Morton G J. Hypothalamic leptin regulation of energy homeostasis and glucose metabolism. J Physiol 583: 437-443, 2007.
25. Morton G J, Thatcher B S, Reidelberger R D, Ogimoto K, Wolden-Hanson T, Baskin D G, Schwartz M W and Blevins J E. Peripheral oxytocin suppresses food intake and causes weight loss in diet-induced obese rats. Am J Physiol Endocrinol Metab 302: E134-E144, 2012.
26. Muttenthaler M, Andersson A, de Araujo A D, Dekan Z, Lewis R J and Alewood P F. Modulating oxytocin activity and plasma stability by disulfide bond engineering. J Med Chem 53: 8585-8596, 2010.
27. Nuevo R, Chatterji S, Fraguas D, Verdes E, Naidoo N, Arango C and yuso-Mateos J L. Increased risk of diabetes mellitus among persons with psychotic symptoms: results from the WHO World Health Survey. J Clin Psychiatry 72: 1592-1599, 2011.
28. Paolisso G, Sgambato S, Giugliano D, Pizza G, Tesauro P, Varricchio M and D'Onofrio F. Effects of oxytocin delivery on counter-regulatory hormone response in insulin-dependent (type 1) diabetic subjects. Horm Res 31: 250-255, 1989.
29. Paolisso G, Sgambato S, Passariello N, Torella R, Giugliano D, Mignano S, Varricchio M and D'Onofrio F. Pharmacological doses of oxytocin affect plasma hormone levels modulating glucose homeostasis in normal man. Horm Res 30: 10-16, 1988.
30. Peet M. Diet, diabetes and schizophrenia: review and hypothesis. Br J Psychiatry Suppl 47: S102-S105, 2004.
31. Purkayastha S, Zhang H, Zhang G, Ahmed Z, Wang Y and Cai D. Neural dysregulation of peripheral insulin action and blood pressure by brain endoplasmic reticulum stress. Proc Natl Acad Sci USA 108: 2939-2944, 2011.
32. Purnell J Q and Flum D R. Bariatric surgery and diabetes: who should be offered the option of remission? JAMA 301: 1593-1595, 2009.
33. Schauer P R, Kashyap S R, Wolski K, Brethauer S A, Kirwan J P, Pothier C E, Thomas S, Abood B, Nissen S E and Bhatt D L. Bariatric surgery versus intensive medical therapy in obese patients with diabetes. N Engl J Med 366: 1567-1576, 2012.
34. Soloff M S, Alexandrova M and Fernstrom M J. Oxytocin receptors: triggers for parturition and lactation? Science 204: 1313-1315, 1979.
35. Takayanagi Y, Kasahara Y, Onaka T, Takahashi N, Kawada T and Nishimori K. Oxytocin receptor-deficient mice developed late-onset obesity. Neuroreport 19: 951-955, 2008.
36. Yamanaka T, Hase S, Sakakibara S, Schwartz I L, Dubois B M and Walter R. Crystalline deamino-dicarba-oxytocin. Preparation and some pharmacological properties. Mol Pharmacol 6: 474-480, 1970.
37. Zhang G, Bai H, Zhang H, Dean C, Wu Q, Li J, Guariglia S, Meng Q and Cai D. Neuropeptide exocytosis involving synaptotagmin-4 and oxytocin in hypothalamic programming of body weight and energy balance. Neuron 69: 523-535, 2011.
38. Zhang G and Cai D. Circadian intervention of obesity development via resting-stage feeding manipulation or oxytocin treatment. Am J Physiol Endocrinol Metab 301: E1004-E1012, 2011.
39. Zhang X, Zhang G, Zhang H, Karin M, Bai H and Cai D. Hypothalamic I KKbeta/NF-kappaB and ER stress link overnutrition to energy imbalance and obesity. Cell 135: 61-73, 2008.

1. A method for treating or preventing diabetes in a subject comprising administering to the subject an amount of oxytocin, or an amount of an oxytocin analog, effective to treat or prevent diabetes.

2. A method for increasing insulin secretion in a subject comprising administering to the subject an amount of oxytocin, or an amount of an oxytocin analog, effective to increase pancreatic insulin secretion.

3. The method of claim 1, wherein the oxytocin or oxytocin analog is administered so as to permit the oxytocin or oxytocin analog to enter the central nervous system of the subject

4. The method of claim 1, wherein the amount of oxytocin or of the oxytocin analog is effective to reduce insulin-resistance.

5. The method of claim 1, wherein the amount of oxytocin or of the oxytocin analog is effective to reduce glucose-intolerance.

6. The method of claim 1, wherein the diabetes is type-1 diabetes.

7. The method of claim 1, wherein the diabetes is type-2 diabetes.

8. (canceled)

9. The method of claim 1, wherein the oxytocin analog is administered.

10. The method of claim 1, wherein the oxytocin is administered.

11. The method of claim 1, wherein the subject is not obese.

12. The method of claim 1, wherein the subject is obese.

13. The method of claim 1, wherein the oxytocin or oxytocin analog is administered directly into the central nervous system of the subject.

14. The method of claim 1, wherein the oxytocin or oxytocin analog is administered intrathecally.

15-16. (canceled)

17. The method of claim 1, wherein the oxytocin or oxytocin analog is administered intranasally.

18. The method of claim 1, wherein the oxytocin or oxytocin analog is administered peripherally to the subject.

19-23. (canceled)

24. A method of improving pancreatic beta cell function in a diabetic subject or in a pre-diabetic subject having impaired beta-cell function comprising administering to the subject an amount of oxytocin or of an oxytocin analog effective to improve pancreatic beta cell function.

25. The method of claim 24, wherein the subject has impaired pancreatic beta cell function.

26. The method of claim 24, wherein the oxytocin or oxytocin analog reduces impairment of pancreatic beta cell insulin secretion.

27. The method of claim 24, wherein the oxytocin or oxytocin analog improves glucose intolerance in the diabetic subject.

28. The method of claim 24, wherein the diabetic subject has type-1 diabetes.

29-32. (canceled)
